(19) United States
(12) Patent Application Publication
(10) Pub. No.: US 2017/0181608 A1
(43) Pub. Date: Jun. 29, 2017

(54) MEDICAL INSTRUMENT

(71) Applicant: OLYMPUS CORPORATION, Tokyo (JP)

(72) Inventor: Yoshiro OKAZAKI, Tokyo (JP)

(73) Assignee: OLYMPUS CORPORATION, Tokyo (JP)

(21) Appl. No.: 15/457,247

(22) Filed: Mar. 13, 2017

Related U.S. Application Data
(63) Continuation of application No. PCT/JP2014/080495, filed on Nov. 18, 2014.

(51) Int. Cl. A61B 1/00; A61B 1/012; A61B 1/005

(52) U.S. Cl. CPC A61B 1/00154; A61B 1/00128; A61B 1/005; A61B 1/012

(57) ABSTRACT

A medical instrument including: an elongated insertion portion having flexibility and being insertable into a living body; a suctional attachment section provided on an outer circumferential surface of the insertion portion at an intermediate position in a longitudinal direction of the insertion portion and having a suction opening at an outer side in a radial direction relative to the insertion portion, opening in the radial direction, and suctionally attaching tissue in the living body; and a rotation mechanism that attaches the suctional attachment section to the insertion portion in such a manner as to be rotatable about an axis in a direction substantially the same as the radial direction.

### CROSS-REFERENCE TO RELATED APPLICATIONS

This is a continuation of International Application PCT/JP 2014/080495, with an international filing date of Nov. 18, 2014, which is hereby incorporated by reference herein in its entirety. This application claims the benefit of International Application PCT/JP2014/080495.

### TECHNICAL FIELD

The present invention relates to a medical instrument.

### BACKGROUND ART

In the related art, there are known cardiac surgery methods in which a medical instrument such as a catheter, an endoscope or the like is inserted into the pericardial cavity through the pericardium from below the xiphoid process, and treatment is performed on the heart in the pericardial cavity from the outside (for example, see Patent Literature 1).

### CITATION LIST

### Patent Literature

{PTL 1}

U.S. Patent Application No. 2004/0064138, Specification

### SUMMARY OF INVENTION

### Technical Problem

The movement directions of a medical instrument in the flat pericardial cavity between the heart and the pericardium are restricted to directions along the surface of the heart. Because the medical instrument inserted into the pericardial cavity is secured at two positions, namely, on the body surface and the pericardium, the possible movements of the medical instrument inside the pericardial cavity are restricted to advancing/retreating movements in the longitudinal direction, rotation movements about the longitudinal axis, and bending movements of a bending section provided at the distal end. Under such restrictions, the treatable area may be limited because the regions in the heart surface that the tip of the medical instrument can approach are limited.

An object of the present invention is to provide a medical instrument that can easily approach an arbitrary position in the heart, inside the pericardial cavity.

### Solution to Problem

An aspect of the present invention is a medical instrument comprising: an elongated insertion portion having flexibility and being insertable into a living body; a suctional attachment section provided on an outer circumferential surface of the insertion portion at an intermediate position in a longitudinal direction of the insertion portion and having a suction opening at an outer side in a radial direction relative to the insertion portion, opening in the radial direction, and suctionally attaching tissue in the living body; and a rotation mechanism that attaches the suctional attachment section to the insertion portion in such a manner as to be rotatable about an axis in a direction substantially the same as the radial direction.

In the above-described aspect, the rotation mechanism may include a circular-ring-shaped fixed ring that is fixed relative to the insertion portion; the suctional attachment section may include a circular-ring-shaped rotating ring disposed coaxially with the fixed ring; one of the fixed ring and the rotating ring may have a circular-ring-shaped recessed portion extending in the circumferential direction on an inner circumferential face or an outer circumferential face thereof; and the other one of the fixed ring and the rotating ring may have a protrusion that is fitted, in a snap-fit manner, into the recessed portion in the outer circumferential face or the inner circumferential face.

In the above-described aspect, the insertion portion may have a bendable bending section closer to a distal end than the suctional attachment section is.

The above-described aspect may further comprise a rotational movement mechanism that moves the position of the suctional attachment section in the circumferential direction of the insertion portion relative to the insertion portion.

The above-described aspect may further comprise a forward/backward movement mechanism that moves the position of the suctional attachment section in the longitudinal direction of the insertion portion relative to the insertion portion.

In the above-described aspect, the medical instrument may be an endoscope.

In the above-described aspect, the medical instrument may be a cylindrical guide sheath into which another medical instrument can be inserted.

In the above-described aspect, the medical instrument may be a cylindrical cover sheath that can be attached to and removed from an outer circumferential surface of an insertion portion of another medical instrument.

### BRIEF DESCRIPTION OF DRAWINGS

FIG. 1 is a diagram showing the overall configuration of a medical instrument according to an embodiment of the present invention.

FIG. 2 is a cross-sectional view taken through I-I in the medical instrument in FIG. 1.

FIG. 3A is a diagram for explaining the method of using the medical instrument in FIG. 1.

FIG. 3B is a diagram for explaining the method of using the medical instrument in FIG. 1.

FIG. 3C is a diagram for explaining the method of using the medical instrument in FIG. 1.

FIG. 4 is a longitudinal sectional view showing a modification of a suctional attachment section in FIG. 1.

FIG. 5 is a longitudinal sectional view showing another modification of the suctional attachment section in FIG. 1.

FIG. 6 is a longitudinal sectional view showing a modification of a suction tube in FIG. 1.

FIG. 7 is a side view showing one modification of the medical instrument in FIG. 1.

FIG. 8 is a longitudinal sectional view showing another modification of the medical instrument in FIG. 1.

FIG. 9 is a longitudinal sectional view showing another modification of the medical instrument in FIG. 1.

FIG. 10A is a side view showing another modification of the suctional attachment section in FIG. 1.

FIG. 10B is a lateral sectional view taken through II-II in the medical instrument in FIG. 10A.

FIG. 11A is a longitudinal sectional view showing another modification of the suctional attachment section and a rotation mechanism in FIG. 1.

FIG. 11B is a lateral sectional view taken through III-III in the medical instrument in FIG. 11A.

FIG. 12A is a diagram for explaining a method of using another modification of the medical instrument in FIG. 1.

FIG. 12B is a diagram for explaining a method of using another modification of the medical instrument in FIG. 1.

FIG. 12C is a diagram for explaining a method of using another modification of the medical instrument in FIG. 1.

FIG. 12D is a diagram for explaining a method of using another modification of the medical instrument in FIG. 1.

### DESCRIPTION OF EMBODIMENT

A medical instrument according to an embodiment of the present invention will be described below with reference to the drawings.

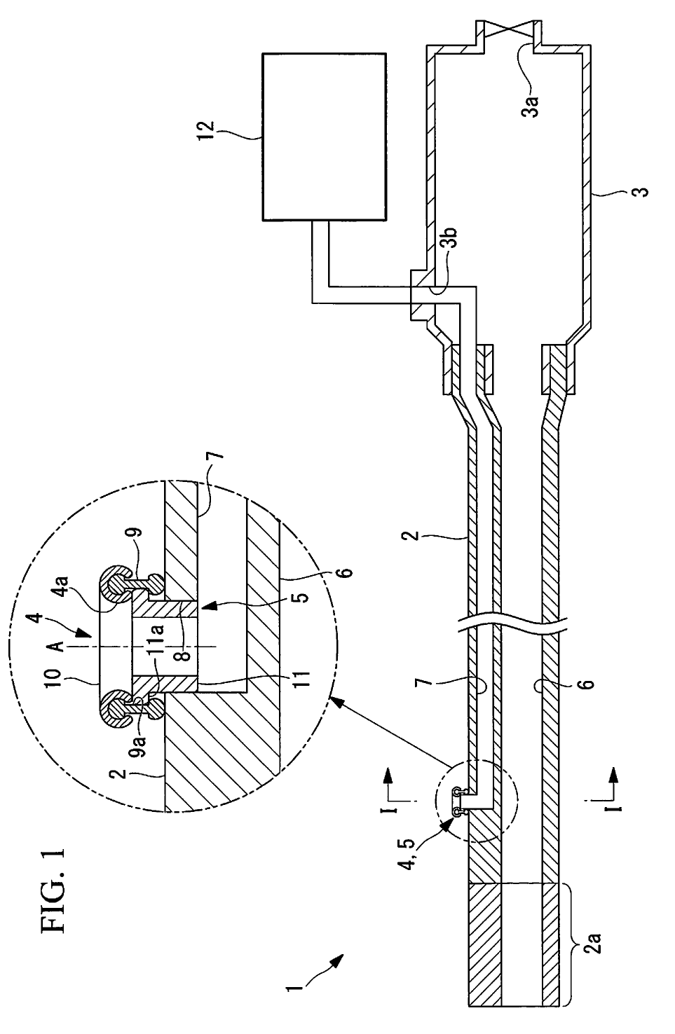

The medical instrument according to this embodiment is a guide sheath 1 for guiding an endoscope inside the body and, as shown in FIG. 1, includes an elongated cylindrical insertion portion 2 having flexibility, an operating section 3 connected to the proximal end of the insertion portion 2, a suctional attachment section 4 provided at an intermediate position in the longitudinal direction of the insertion portion 2, and a rotation mechanism 5 for attaching the suctional attachment section 4 to the insertion portion 2 in a manner allowing rotation thereof.

As shown in FIG. 1, the insertion portion 2 includes a bendable bending section 2a at the distal end section, and the distal end face can be pointed in any direction by bending the bending section 2a.

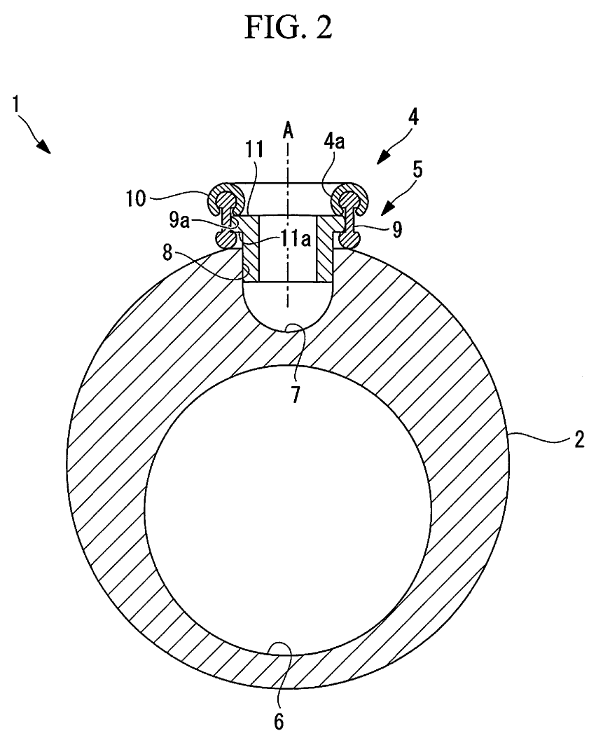

As shown in FIG. 2, the insertion portion 2 has a channel 6 and a suction tube 7 formed parallel to each other along the longitudinal direction. The channel 6, which is a space for inserting the endoscope, penetrates from the distal end face of the insertion portion 2 to the proximal end face thereof and communicates with an introduction port 3a provided in the operating section 3 via the interior of the operating section 3. The suction tube 7 allows an opening 8 provided in the insertion portion 2 to communicate with a suction port 3b provided in the operating section 3. The opening 8 opens in the radial direction in the outer circumferential face of the insertion portion 2, closer to the proximal end than the bending section 2a is.

As shown in FIG. 1 and FIG. 2, the suctional attachment section 4 includes a circular-ring-shaped rotating ring 9 and a ring-shaped pad 10 that is attached to one end portion of the rotating ring 9 in the central axis direction thereof.

The rotating ring 9 has an inner diameter dimension larger than the inner diameter dimension of the opening 8. A recessed portion 9a formed around the entire circumference in the circumferential direction is provided on the inner circumferential surface of the rotating ring 9.

The pad 10 is formed of an elastic material such as silicon rubber, for example, and is attached to the end portion so as to cover the end portion of the rotating ring 9, which is positioned on the outer side in the radial direction relative to the insertion portion 2.

The rotation mechanism 5 includes a circular-ring-shaped fixed ring 11. One end portion in the center axis direction of the fixed ring 11 is fitted inside the opening 8, and is secured to the inner circumferential surface of the opening 8. The other end portion of the fixed ring 11 protrudes in the radial direction of the insertion portion 2 to the outer side of the insertion portion 2 from the opening 8, and a flange-shaped protrusion 11a that protrudes outward in the radial direction thereof and that can be inserted inside the recessed portion 9a is provided in the outer circumferential surface thereof.

The rotating ring 9 having the recessed portion 9a constitutes a female snap, the fixed ring 11 having the protrusion 11a constitutes a male snap, and the protrusion 11a is fitted into the recessed portion 9a in a snap-fit manner. Accordingly, the rotating ring 9 and the fixed ring 11 are coaxially connected, and the protrusion 11a can move in the circumferential direction inside the recessed portion 9a, thereby being capable of relative rotation about the center axis A.

The space formed at the inner side of the pad 10 forms a suction opening 4a that communicates with the opening 8 in the radial direction of the insertion portion 2. When suction is applied to the interior of the suction tube 7 by operating a suction apparatus 12 connected to the suction port 3b in a state in which the pad 10 is disposed opposing an object, negative pressure is generated in the suction opening 4a, and the object is suctionally attached to the suction opening 4a. At this time, by closely contacting the pad 10 with the surface of the object, the suction opening 4a is sealed off by the surface of the object, so that the object becomes securely attached.

Next, the operation of the guide sheath 1 having such a configuration will be described.

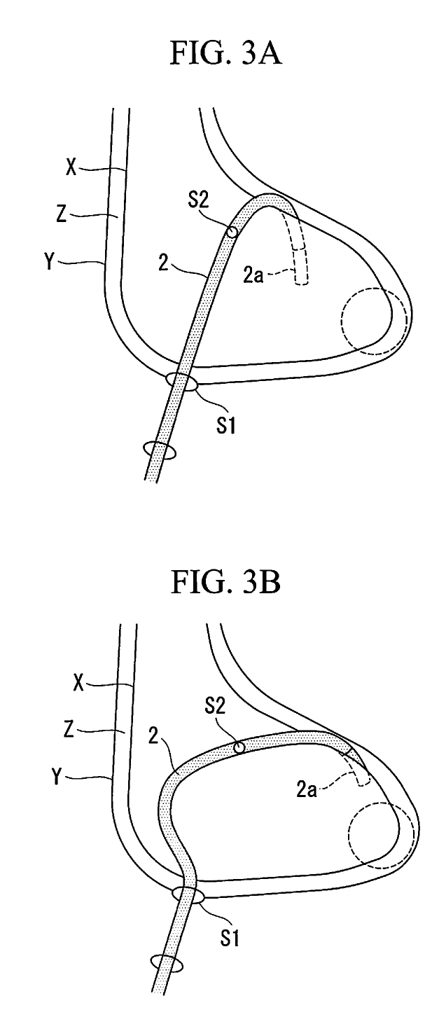

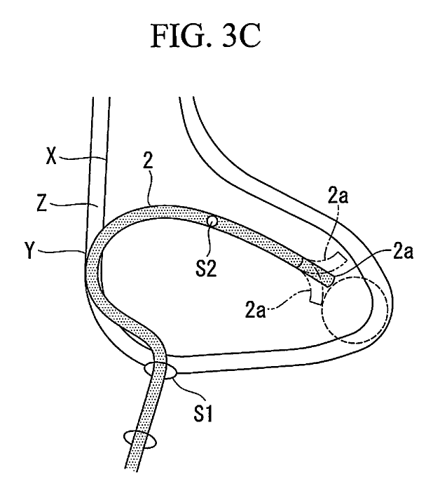

To perform observation and treatment of the heart X with an endoscope using the guide sheath 1 according to this embodiment, as shown in FIG. 3A, in a state in which the suction apparatus 12 is stopped, the insertion portion 2 of the guide sheath 1 is introduced into the pericardial cavity Z through the pericardium Y from the surface of the body below the xiphoid process, and the insertion portion 2 is inserted to a position where the distal end of the insertion portion 2 goes behind the heart X to the opposite side thereof. Next, negative pressure is generated in the suction opening 4a by operating the suction apparatus 12, so that the surface of the heart X becomes attached to the suctional attachment section 4. Accordingly, an intermediate position in the longitudinal direction of the insertion portion 2 is secured to the heart X. In this state, the position of the insertion portion 2 relative to the heart X is fixed in the radial direction at the portion where it penetrates the pericardium Y, and is fixed in the longitudinal direction and the radial direction at the suctional attachment section 4.

Next, in the state where the suctional attachment section 4 is attached to the surface of the heart X, as shown in FIG. 3B, when the insertion portion 2 is pushed in, the insertion portion 2 moves with the section where it penetrates the pericardium Y and the suctional attachment section 4 serving as fulcrums S1 and S2, respectively. In other words, the insertion portion 2 bends between the fulcrum S1 and the fulcrum S2. At this time, at the fulcrum S2, the suctional attachment section 4 and the insertion portion 2 are rotatable about an axis approximately in the direction of the normal to the surface of the heart X. Therefore, the section of the insertion portion 2 closer to the distal end than the fulcrum S2 rotationally moves along the surface of the heart X about the fulcrum S2 by means of an elastic restoring force of the insertion portion 2 which tends to return it to a substantially linear shape. Accordingly, the section of the insertion portion 2 positioned inside the pericardial cavity Z bends in an approximate C-shape as a whole, and the distal end of the insertion portion 2 also rotationally moves along an approximately arc-shaped path centered on the fulcrum S1.

When the insertion portion 2 is pushed in farther from the state shown in FIG. 3B, the section of the insertion portion 2 closer to the distal end than the fulcrum S2 further rotationally moves about the fulcrum S2, and the section of the insertion portion 2 positioned inside the pericardial cavity Z bends to assume an approximate U-shape with an even larger curvature as a whole, and the distal end of the insertion portion 2 also rotationally moves further. Accordingly, as shown in FIG. 3C, the distal end of the insertion portion 2 can reach the cardiac apex, which is located at the side of the fulcrum S1 (the part enclosed by the circle). In this state, by bending the bending section 2a, fine positioning of the distal end of the insertion portion 2 relative to the treatment site is performed.

Next, by inserting the endoscope into the pericardial cavity Z via the guide sheath 1, observation and treatment of the treatment site in the heart X can be performed with the endoscope.

Thus, with this embodiment, by pushing in the insertion portion 2 in a state in which the suctional attachment section 4 provided at an intermediate position in the longitudinal direction of the insertion portion 2 is secured to the surface of the heart X, the entire section of the insertion portion 2 positioned inside the pericardial cavity Z is bent by a large amount, and the distal end of the insertion portion 2 rotationally moves by a large amount along the surface of the heart X, with the suctional attachment section 4 serving as the center of rotation. The amount of movement of the distal end of the insertion portion 2 at this time can be easily controlled by changing the position at which the suctional attachment section 4 is secured to the heart X and the amount by which the insertion portion 2 is pushed in. Accordingly, an advantage is afforded in that the distal end of the insertion portion 2 can be made to easily approach an arbitrary position in a wide area on the surface of the heart X, including positions that have been difficult to approach conventionally, such as the cardiac apex.

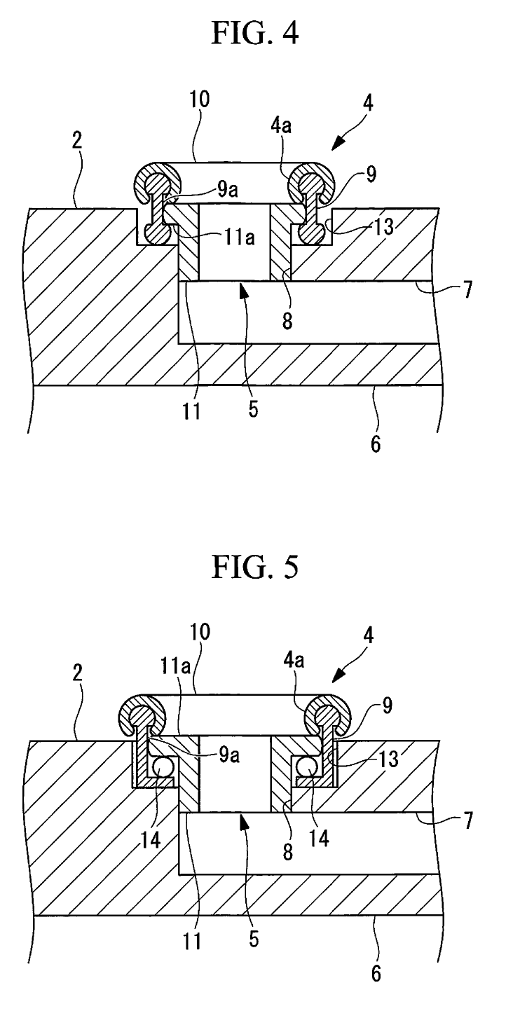

In this embodiment, as shown in FIG. 4, a countersunk-hole-like recessed portion 13 may be formed around the opening 8 in the outer circumferential surface of the insertion portion 2, and part of the rotating ring 9 may be accommodated inside the recessed portion 13.

By doing so, the amount by which the rotating ring 9 protrudes from the outer circumferential surface of the insertion portion 2 can be made small.

In this embodiment, as shown in FIG. 5, a ball bearing 14 may be provided between the fixed ring 11 and the rotating ring 9.

By doing so, the rotational movement of the rotating ring 9 relative to the fixed ring 11 can be made more smooth.

Although this embodiment has been explained in terms of the guide sheath 1 serving as a medical instrument, instead of this, the suctional attachment section 4 and the rotation mechanism 5 may be provided directly on an endoscope.

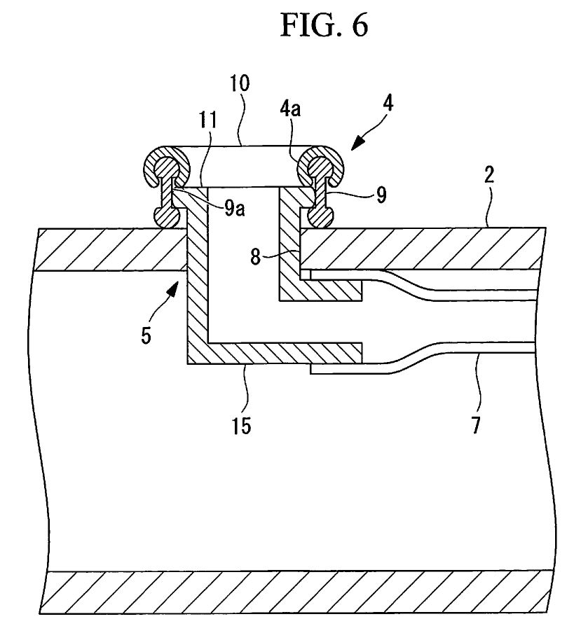

In this case, the configuration of the suctional attachment section 4, the rotation mechanism 5, the suction tube 7, and the opening 8 could be used as is in the insertion portion of the endoscope. Alternatively, as shown in FIG. 6, a member such as a PTFE tube may be used as the suction tube 7. Reference sign 15 is a joint pipe that connects the rotating ring 9 and the suction tube 7, and part of the joint pipe 15 constitutes the fixed ring 11 described above.

In the case where the endoscope is inserted into the insertion portion 2 of the guide sheath 1 which has been positioned inside the pericardial cavity Z, as shown in FIG. 3C, as the endoscope is inserted, the bending stiffness of the insertion portion 2 as a whole changes, and the insertion portion 2 can rotationally move about the fulcrum S2. In contrast, in the case where the suctional attachment section 4 and the rotation mechanism 5 are provided on the insertion portion of the endoscope, the distal end of the insertion portion of the endoscope can be directly positioned relative to the desired site in the heart X, and therefore, this drawback does not occur.

In this embodiment, a forward/backward movement mechanism that moves the suctional attachment section 4 and the rotation mechanism 5 in the longitudinal direction of the insertion portion 2 may be provided.

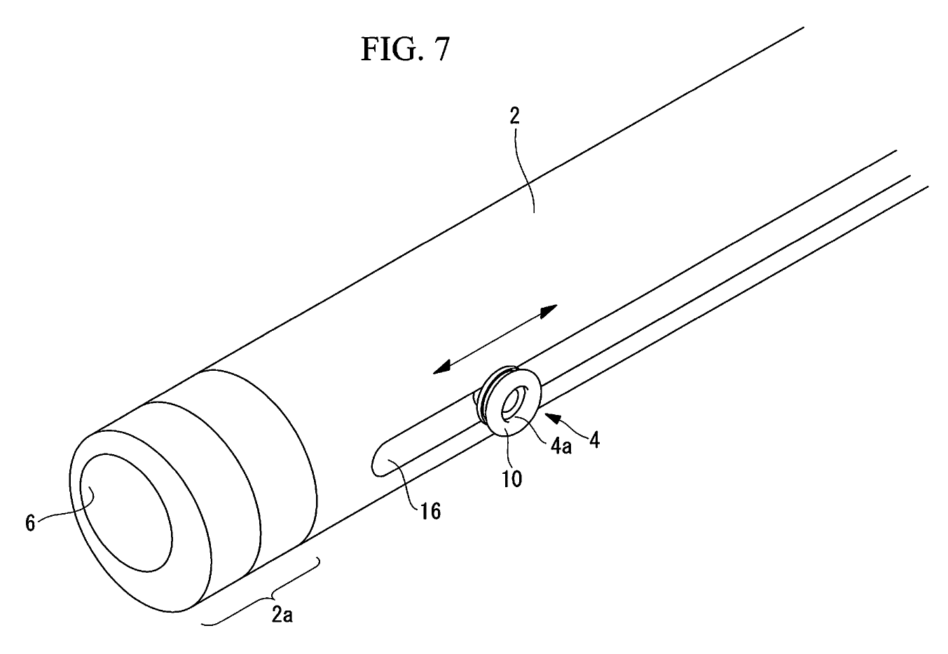

As the forward/backward movement mechanism, as shown in FIG. 7, for example, a groove 16 in the longitudinal direction is formed in the outer circumferential surface of the insertion portion 2, so that the suctional attachment section 4 and the rotation mechanism 5 are provided in a manner capable of translational movement, as a single unit, along the groove 16. The positioning of the suctional attachment section 4 and the rotation mechanism 5 in the groove 16 can be performed at the operating section 3.

By doing so, it is possible to arbitrarily change the position of the fulcrum S2 when rotationally moving the insertion portion 2 inside the pericardial cavity Z, and it is possible to further enlarge the area that the distal end of the insertion portion 2 can approach.

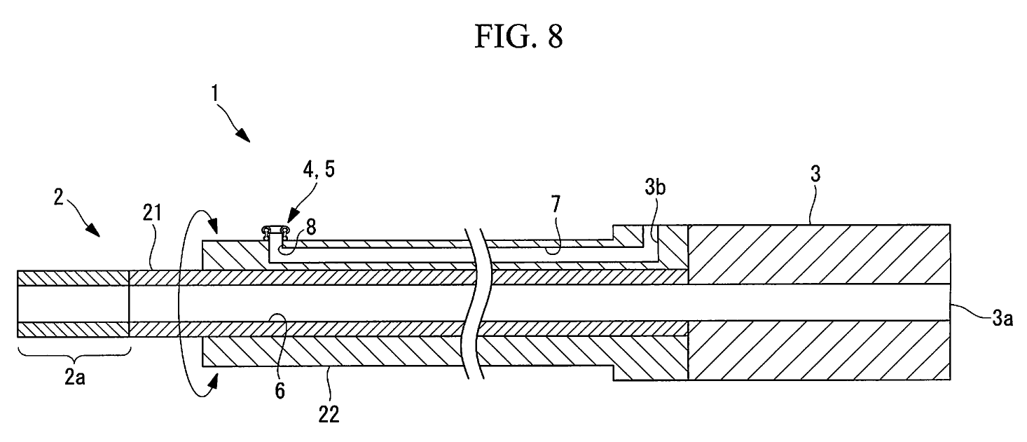

In this embodiment, as shown in FIG. 8, a rotational movement mechanism that moves the suctional attachment section 4 and the rotation mechanism 5 in the circumferential direction of the insertion portion 2 may be provided.

In this case, the insertion portion 2 includes a cylindrical body 21 and a cylindrical rotating pipe 22 attached to the outer side of the body 21 in a manner capable of rotating about the longitudinal axis relative to the body, and the suction tube 7, the suctional attachment section 4, and the rotation mechanism 5 are provided on the rotating pipe 22. The rotational movement mechanism is formed of the rotating pipe 22.

By doing so, to dispose the suctional attachment section 4 so as to oppose the heart X, the insertion portion 2 as a whole is not twisted but only the rotating pipe 22 need be rotated relative to the body 21; therefore, the operation can be simplified. In addition, by rotating the rotating pipe 22 in the state in which the body 21 is stationary, it is possible to easily change the tissue to which the suctional attachment section 4 is attached, between the heart X and the pericardium Y. In the state in which the heart X or the pericardium Y is attached to the suctional attachment section 4, the body 21 can be rotated about the longitudinal axis inside the rotating pipe 22, and the ease of use can be improved.

The rotating pipe 22 may constitute a cover sheath that can be attached to and removed from the insertion portion 2, as a single unit. By attaching a cover sheath to a general-purpose guide sheath 1 or the medical instrument, such as the endoscope, a general-purpose medical instrument can also be operated, as shown in FIGS. 3A to 3C.

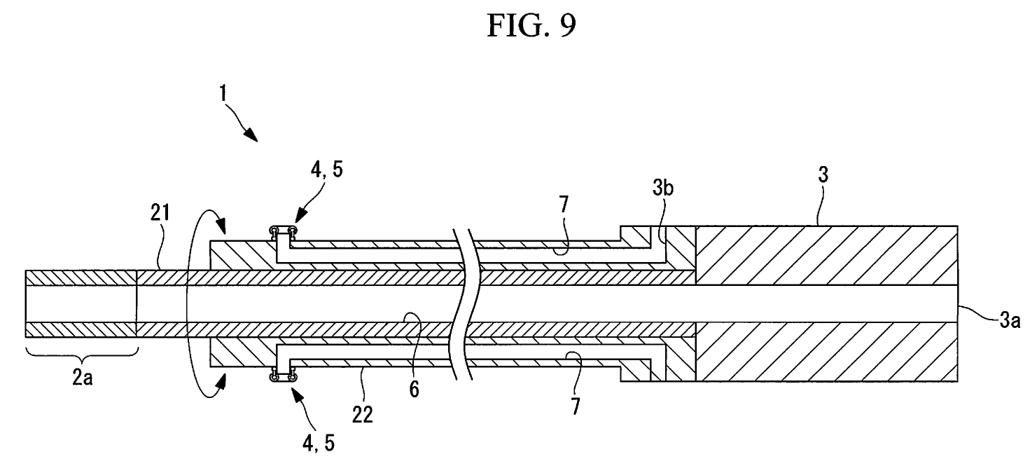

In this embodiment, a plurality of sets of the suctional attachment sections 4 and the rotation mechanisms 5 may be provided with gaps therebetween in the circumferential direction of the insertion portion 2. In the example shown in FIG. 9, two sets of suctional attachment sections 4 and rotation mechanisms 5 are provided in the rotating pipe 22, symmetrically with respect to the longitudinal axis of the insertion portion 2.

By doing so, one of the suctional attachment sections 4 can be attached to the heart X, and the other suctional attachment section 4 can be attached to the pericardium Y at the same time, and thus, the securing force of the insertion portion 2 can be increased.

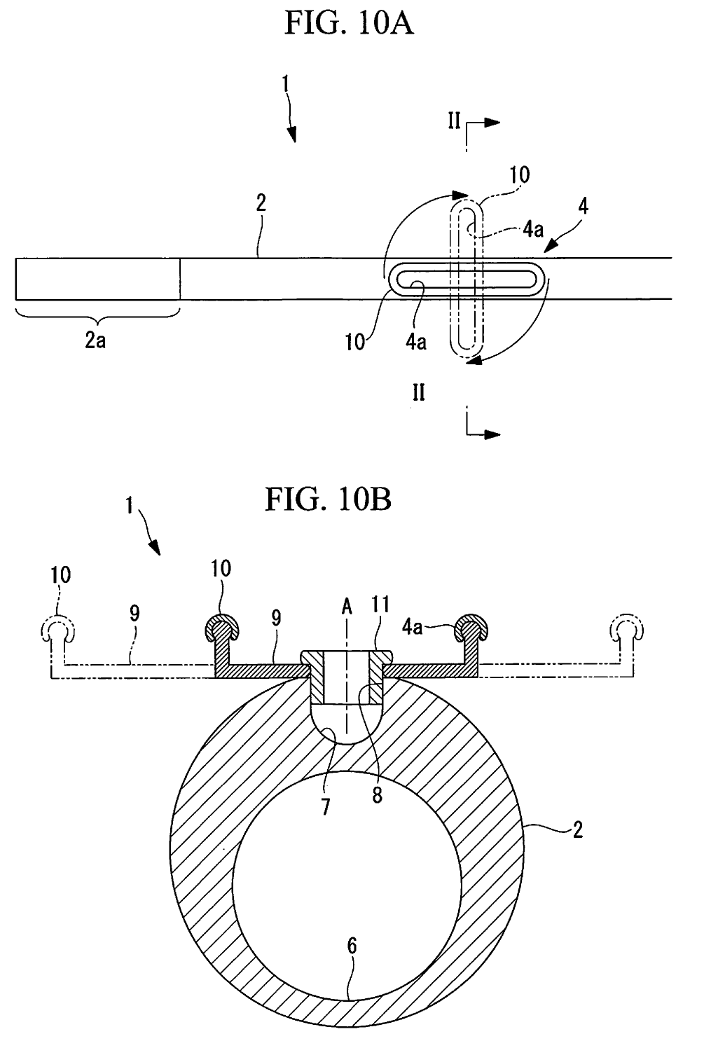

In this embodiment, as shown in FIG. 10A and FIG. 10B, the rotating ring 9 may have an elliptical ring shape. By doing so, the suction area of the rotating ring 9 can be increased, so that the suctional attachment section 4 can be secured to the heart X or the pericardium Y with a stronger suction force. When the insertion portion 2 is moved inside the body, by disposing the longitudinal axis of the rotating ring 9 so as to be parallel to the longitudinal direction of the insertion portion 2, it is possible to prevent the rotating ring 9 from hindering the manipulation of the insertion portion 2.

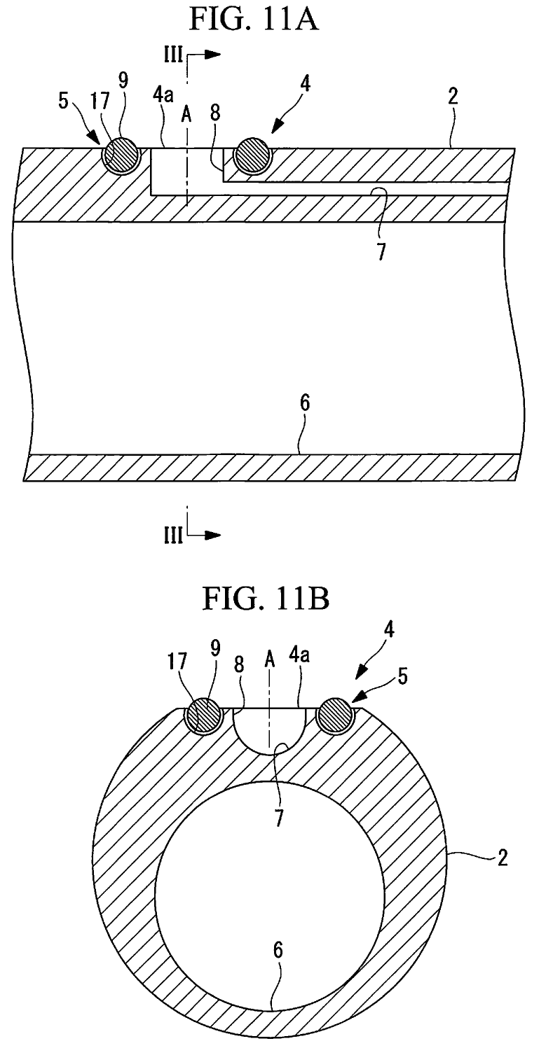

In this embodiment, as shown in FIG. 11A and FIG. 11B, the rotating ring 9 may be directly provided on the outer circumferential surface of the insertion portion 2. By doing so, it is possible to reduce the number of parts.

In this case, instead of the fixed ring 11, a circular ring-shaped groove (rotating mechanism) 17 formed around the opening 8 may be provided in the outer circumferential surface of the insertion portion 2, and the rotating ring 9 is inserted into the groove 17 so as to be able to rotate in the circumferential direction. One end portion of the rotating ring 9 in the center axis direction protrudes from the groove 17 and can thus contact the tissue. So that the rotating ring 9 smoothly rotates inside the groove 17, it is preferable to apply a lubricant having high biocompatibility, such as silicone oil, between the outer circumferential surface of the rotating ring 9 and the inner circumferential surface of the groove 17.

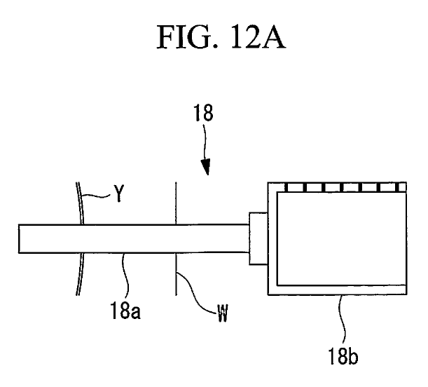

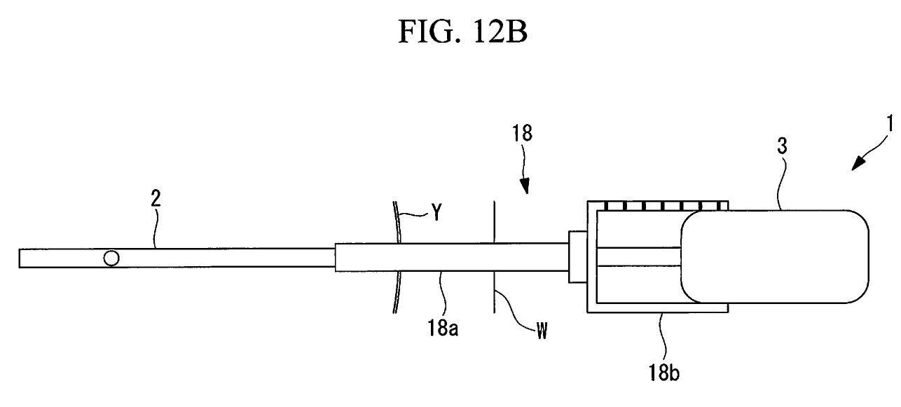

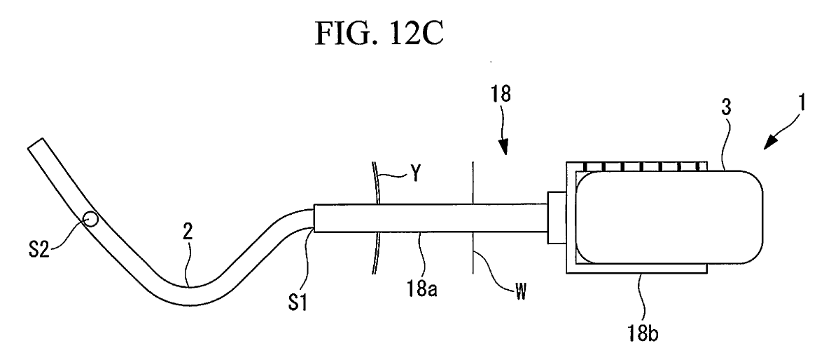

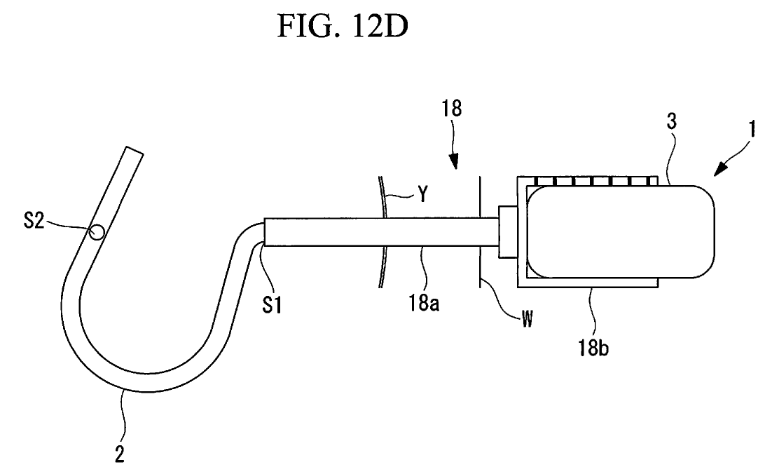

In this embodiment, as shown in FIGS. 12A to 12D, a measurement instrument 18 for measuring the amount of insertion of a medical instrument, for example, the insertion portion 2 of the endoscope 1, into the pericardial cavity Z may be provided.

The measurement instrument 18 includes a sheath 18a that can be percutaneously inserted into the pericardial cavity Z and a measurement unit 18b that is connected to the proximal end of the sheath 18a. The measurement unit 18b is formed of a transparent material, is a cylindrical container that communicates with the inside of the sheath 18a, and accommodates the operating section 3 of the endoscope 1 in which the insertion portion 2 is inserted into the body via the measurement unit 18b and the inside of sheath 18a.

A scale is provided in the measurement unit 18b so that the amount of insertion of the operating section 3 relative to the measurement unit 18b can be quantitatively detected on the basis of the scale. In addition, a scale is also provided in the longitudinal direction on the outer circumferential surface of the sheath 18a, and the amount of insertion of the sheath into the living body can be quantitatively detected on the basis of the scale.

The sheath 18a has higher rigidity than the insertion portion 2. Thus, when the insertion portion 2 of the endoscope 1 is made to protrude from the distal end of the sheath 18a inserted into the pericardial cavity Z, and the insertion portion 2 of the endoscope 1 is made to bend similarly to FIGS. 3A to 3C, the insertion portion 2 bends with the distal end of the sheath 18a serving as the fulcrum S1, not the portion where it penetrates the pericardium Y. Therefore, the bending shape of the insertion portion 2 can be controlled using not only the amount of insertion of the insertion portion 2 into the pericardial cavity Z but also the amount of insertion of the sheath 18a into the pericardial cavity Z, so that the distal end position of the insertion portion 2 can be manipulated.

During surgery, in the case of repeated insertion and removal of the insertion portion 2 into the pericardial cavity Z via the sheath 18a, it is preferable that the position of the distal end of the insertion portion 2 when inserted into the pericardial cavity Z be the same each time. Thus, the amount of insertion of the sheath 18a with respect to the body surface W and the amount of insertion of the insertion portion 2 with respect to the sheath 18a, are quantitatively recorded in advance. Then, the next time the insertion portion 2 is inserted into the pericardial cavity Z, by disposing the sheath 18a and the insertion portion 2 so that the amount of insertion of the sheath 18a with respect to the body surface W and the amount of insertion of the insertion portion 2 with respect to the sheath 18a take the recorded values, the distal end of the insertion portion 2 can be disposed at the same position in the pericardial cavity Z as that position used the previous time.

As a result, the following aspect is read by the above described embodiment of the present invention.

An aspect of the present invention is a medical instrument comprising: an elongated insertion portion having flexibility and being insertable into a living body; a suctional attachment section provided on an outer circumferential surface of the insertion portion at an intermediate position in a longitudinal direction of the insertion portion and having a suction opening at an outer side in a radial direction relative to the insertion portion, opening in the radial direction, and suctionally attaching tissue in the living body; and a rotation mechanism that attaches the suctional attachment section to the insertion portion in such a manner as to be rotatable about an axis in a direction substantially the same as the radial direction.

With this aspect, in a state in which the insertion portion is inserted into the pericardial cavity from the body surface through the pericardium, by disposing the insertion portion so that the suction opening is blocked by the heart or the pericardium and by suctionally attaching the heart or pericardium to the suction opening, the intermediate position in the longitudinal direction of the insertion portion is fixed to the heart or the pericardium. In this state, when the insertion portion is pushed in further inside the pericardial cavity, the insertion portion bends between the portion where it penetrates the pericardium and the suctional attachment section.

At this time, the suctional attachment section and the insertion portion can be relatively rotated about an axis in a direction substantially normal to the surface of the heart by the rotation mechanism, whereby the section of the insertion portion closer to the distal end than the suctional attachment section is rotationally moved about the suctional attachment section along the surface of the heart, and the section of the insertion portion disposed inside the pericardial cavity bends in an approximate C-shape or an approximate U-shape overall. The bending shape of the insertion portion is controlled by the attachment position of the heart by the suctional attachment section and the amount by which the insertion portion is pushed into the pericardial cavity. Accordingly, by enabling bending movement in addition to advancing/retreating movement in the longitudinal direction of the insertion portion, the area that the distal end of the insertion portion can approach in the pericardial cavity becomes larger, and the distal end of the insertion portion can be easily made to approach an arbitrary position on the surface of the heart.

In the above-described aspect, the rotation mechanism may include a circular-ring-shaped fixed ring that is fixed relative to the insertion portion; the suctional attachment section may include a circular-ring-shaped rotating ring disposed coaxially with the fixed ring; one of the fixed ring and the rotating ring may have a circular-ring-shaped recessed portion extending in the circumferential direction on an inner circumferential face or an outer circumferential face thereof; and the other one of the fixed ring and the rotating ring may have a protrusion that is fitted, in a snap-fit manner, into the recessed portion in the outer circumferential face or the inner circumferential face.

By doing so, the rotating ring can be coupled, in a snap-fit and simple manner, to the fixed ring which is fixed to the insertion portion. The fixed ring and the rotating ring, which are coupled to each other, can be relatively rotated by means of rotational movement of the protrusion in the recessed portion.

In the above-described aspect, the insertion portion may have a bendable bending section closer to a distal end than the suctional attachment section is.

By doing so, it is possible to finely adjust the position of the distal end of the insertion portion relative to the heart by means of the bending movement of the bending section.

The above-described aspect may further comprise a rotational movement mechanism that moves the position of the suctional attachment section in the circumferential direction of the insertion portion relative to the insertion portion.

With this configuration, by rotationally moving the suctional attachment section in the circumferential direction relative to the insertion portion, it is possible to adjust the position of the suctional attachment section in the circumferential direction of the insertion portion to a position where the suction opening opposes the heart or the pericardium, without rotating the entire insertion portion about the longitudinal axis.

The above-described aspect may further comprise a forward/backward movement mechanism that moves the position of the suctional attachment section in the longitudinal direction of the insertion portion relative to the insertion portion.

With this configuration, by moving the suctional attachment section in a straight line in the longitudinal direction relative to the insertion portion and changing the fixed position of the insertion portion relative to the heart or pericardium, it is possible to change the moving area of the distal end of the insertion portion when the insertion portion is rotationally moved about the suctional attachment section.

In the above-described aspect, the medical instrument may be an endoscope.

With this configuration, it is possible to directly fix the endoscope relative to the heart or pericardium by means of the suctional attachment section, and the distal end of the endoscope can be correctly positioned at the desired position on the surface of the heart.

In the above-described aspect, the medical instrument may be a cylindrical guide sheath into which another medical instrument can be inserted.

With this configuration, in the case where a medical instrument, such as an endoscope or treatment tool, is repeatedly inserted into and removed from the pericardial cavity via the guide sheath, the distal end of the insertion portion of the medical instrument can be guided to substantially the same position in the pericardial cavity each time.

In the above-described aspect, the medical instrument may be a cylindrical cover sheath that can be attached to and removed from an outer circumferential surface of an insertion portion of another medical instrument.

With this configuration, by attaching the cover sheath to the insertion portion of a medical instrument, such as a general-purpose endoscope or treatment tool, it is possible to apply the present invention to general-purpose medical instruments.

### REFERENCE SIGNS LIST

1 medical instrument, guide sheath, endoscope
2 insertion portion
2a bending section
3 operating section
4 suctional attachment section
4a suction opening
5 rotation mechanism
6 channel
7 suction tube
8 opening
9 rotating ring
9a recessed portion
10 pad
11 fixed ring
11a protrusion
12 suction apparatus
13 recessed portion
14 ball bearing
15 joint pipe
16 groove (forward/backward movement mechanism)
17 groove
18 measurement instrument
21 body
22 rotating pipe (rotational movement mechanism)

1. A medical instrument comprising:
an elongated insertion portion having flexibility and being insertable into a living body;
a suctional attachment section provided on an outer circumferential surface of the insertion portion at an intermediate position in a longitudinal direction of the insertion portion and having a suction opening at an outer side in a radial direction relative to the insertion portion, opening in the radial direction, and suctionally attaching tissue in the living body; and
a rotation mechanism that attaches the suctional attachment section to the insertion portion in such a manner as to be rotatable about an axis in a direction substantially the same as the radial direction.

2. A medical instrument according to claim 1, wherein
the rotation mechanism includes a circular-ring-shaped fixed ring that is fixed relative to the insertion portion;
the suctional attachment section includes a circular-ring-shaped rotating ring disposed coaxially with the fixed ring;
one of the fixed ring and the rotating ring has a circular-ring-shaped recessed portion extending in the circumferential direction on an inner circumferential face or an outer circumferential face thereof; and
the other one of the fixed ring and the rotating ring has a protrusion that is fitted, in a snap-fit manner, into the recessed portion in the outer circumferential face or the inner circumferential face.

3. A medical instrument according to claim 1, wherein the insertion portion has a bendable bending section closer to a distal end than the suctional attachment section is.

4. A medical instrument according to claim 1, further comprising a rotational movement mechanism that moves the position of the suctional attachment section in the circumferential direction of the insertion portion relative to the insertion portion.

5. A medical instrument according to claim 1, further comprising a forward/backward movement mechanism that moves the position of the suctional attachment section in the longitudinal direction of the insertion portion relative to the insertion portion.

6. A medical instrument according to claim 1, which is an endoscope.

7. A medical instrument according to claim 1, which is a cylindrical guide sheath into which another medical instrument can be inserted.

8. A medical instrument according to claim 1, which is a cylindrical cover sheath that can be attached to and removed from an outer circumferential surface of an insertion portion of another medical instrument.
